(19) United States
(12) Patent Application Publication
(10) Pub. No.: US 2005/0202542 A1
(43) Pub. Date: Sep. 15, 2005

(54) Production method of D-alloisoleucine

(75) Inventors: Shogo Maruyama, Kawasaki-shi (JP); Ikuo Kira, Kawasaki-shi (JP); Tadashi Takemoto, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 10/855,349

(22) Filed: May 28, 2004

(30) Foreign Application Priority Data
Mar. 12, 2004 (JP) 71577/2004

(51) Int. Cl. C12P 13/04

(52) U.S. Cl. 435/106

(57) ABSTRACT

The present invention provides a production method of D-alloisoleucine, a protected compound thereof or a salt thereof, which is industrially useful, and which efficiently produces D-alloisoleucine having a high purity, a protected compound thereof and a salt thereof, and more particularly, a method including steps (a) to (c): (a) carbamoylating L-isoleucine to give L-(N-carbamoyl)isoleucine, (b) conducting intramolecular dehydration condensation of L-(N-carbamoyl)isoleucine to give L-5-sec-butylhydantoin, and (c) conducting an enzyme reaction of L-5-sec-butylhydantoin with hydantoin racemase (HRase), D-hydantoinase (D-HHase) and N-carbamoyl-D-amino acid hydrolase (D-CHase) to give D-alloisoleucine.

### TECHNICAL FIELD OF THE INVENTION

The present invention relates to an industrially useful method capable of efficiently producing D-alloisoleucine having a high purity, a protected compound thereof and a salt thereof.

### BACKGROUND OF THE INVENTION

D-alloisoleucine is useful as a starting material for peptide medicines and the like. The most important aspect in producing D-alloisoleucine is how to afford asymmetry of the carbon atom at the β-position. Generally, D-alloisoleucine is produced using (1) d-2-methyl-butyraldehyde or (2) L-isoleucine as a starting material.

In the method of the above-mentioned (1), d-2-methyl-butyraldehyde is used as an asymmetric source. However, it is an expensive optically active aldehyde, and use thereof is uneconomical. Even if d-2-methyl-butyraldehyde is used, the object substance will be produced by the Strecker method (method using highly toxic hydrogen cyanide and ammonia to give cyanhydrin, followed by hydrolysis thereof, see Chem. Rev. 42(1948), 236) or by the Bucherer-Berg method (hydantoin synthesis using alkaline cyanide and ammonium carbonate, see Chem. Rev. 46(1950), 422-424) to give a substituted hydantoin derivative, followed by hydrolysis thereof. Since the object substance is obtained as a mixture of L-isoleucine and D-alloisoleucine, a particular optically active form alone needs to be separated using acylase and the like. Therefore, since the above-mentioned method (1) not only requires an expensive starting material but also requires use of a reagent difficult to handle and complicated operations, it is not entirely suitable as an industrial method.

L-isoleucine used as an asymmetric source in the above-mentioned method (2) can be obtained at a low cost by a fermentation method and is economical. Therefore, establishment of an efficient industrial production method of D-alloisoleucine having a high purity, a protected compound thereof and a salt thereof, which comprises use of L-isoleucine as a starting material, is expected. This method is generally considered to specifically comprise acetylation of L-isoleucine, then racemization thereof, and reaction with acylase to separate and give only a particular optically active form. However, it may be difficult to say that this method is satisfactory from the aspect of productivity.

### SUMMARY OF THE INVENTION

The present invention aims at providing an industrially useful method that can efficiently produce D-alloisoleucine having a high purity, a protected compound thereof or a salt thereof.

The present inventors have conducted various intensive studies and found that D-alloisoleucine having a high purity, a protected compound thereof and a salt thereof can be produced efficiently by the use of L-isoleucine as a starting material and by going through the following steps (a) to (c), and that this method is industrially useful, and based on these findings, completed the present invention.

Accordingly, the present invention provides the following.

[1] A production method of D-alloisoleucine, a protected compound thereof or a salt thereof, which comprises steps (a) to (c):
(a) carbamoylating L-isoleucine to give L-(N-carbamoyl)isoleucine,
(b) conducting intramolecular dehydration condensation of L-(N-carbamoyl)isoleucine to give L-5-sec-butylhydantoin, and
(c) conducting an enzyme reaction of L-5-sec-butylhydantoin with hydantoin racemase (HRase), D-hydantoinase (D-HHase) and N-carbamoyl-D-amino acid hydrolase (D-CHase) to give D-alloisoleucine.

[2] The above-mentioned production method [1], further comprising protecting the D-alloisoleucine obtained in step (c) to give a protected compound of D-alloisoleucine.

[3] The above-mentioned production method [1], further comprising protecting the D-alloisoleucine obtained in step (c) and forming a salt of the protected compound of D-alloisoleucine to give a salt of the protected compound of D-alloisoleucine.

According to the present invention, D-alloisoleucine having a high purity, a protected compound thereof and a salt thereof can be produced efficiently, and this method is industrially useful.

### BEST MODE FOR EMBODYING THE INVENTION

It is essential that the production method of D-alloisoleucine, a protected compound thereof and a salt thereof of the present invention comprise steps (a) to (c). According to the method of the present invention, an object substance having a high purity can be produced efficiently and this method is industrially useful.

The steps (a) to (c) are explained in detail in the following.

Step (a): A Step of Carbamoylating L-isoleucine to Give L-(N-carbamoyl)isoleucine

L-isoleucine can be carbamoylated by using, for example, alkali metal cyanide. To be precise, alkali metal cyanide is added to L-isoleucine in water to achieve carbamoylation. Alkali metal cyanide can be added as it is or in the form of an aqueous solution. For carbamoylation, since L-isoleucine needs to be dissolved in water at a high concentration, pyridine is generally used. Pyridine here may be used as appropriate in an amount that can dissolve L-isoleucine, and, for example, 1 molar equivalent-3 molar equivalents relative to L-isoleucine is generally added.

Divisional addition (e.g., dropwise addition and the like) of an aqueous solution of alkali metal cyanide can carry out the reaction without pyridine.

As the alkali metal cyanide, for example, sodium cyanide, potassium cyanide and the like can be mentioned and economical sodium cyanide is preferably used. Alkali metal cyanide is used in an amount of generally 0.8 molar equivalent-1.5 molar equivalents, preferably 1.0 molar equivalent-1.2 molar equivalents, relative to L-isoleucine. Alkali metal cyanide may be used in any form of an aqueous solution, a slurry or a solid.

Water to be used as a solvent is generally used in a 1.5 to 20-fold, preferably 2.5- to 10-fold amount, in weight ratio relative to L-isoleucine. This range includes water content in an aqueous solution of alkali metal cyanide, when alkali metal cyanide is used as an aqueous solution.

Carbamoylation of L-isoleucine generally completes at 50-100° C., preferably 60-80° C., generally in 5 min-5 hrs, preferably 10 min-3 hrs.

After the completion of the reaction, the reaction mixture is acidified with an acid, such as hydrochloric acid and the like to allow precipitation of L-(N-carbamoyl)isoleucine as crystals. While L-(N-carbamoyl)isoleucine can be taken out by filtration and the like, it can be used for the next step without taking out.

As L-isoleucine, which is a starting material in the present invention, one having a high purity is commercially available for medicine. In addition, it is generally produced by fermentation methods or protein hydrolysis. Those obtained by these methods concurrently contain branched chain amino acids other than L-isoleucine, which are difficult to separate from L-isoleucine. To improve purity of L-isoleucine by removing other concurrently contained branched chain amino acids, a purification step (e.g., conversion to hydrochloride or aromatic sulfonate or chromatographic separation with resin and the like) is necessary (see JP-A-2001-316342 etc.). In the present invention, however, even if L-isoleucine concurrently containing other branched chain amino acids is used as a starting material, other branched chain amino acids are weeded out while performing steps (a) to (c), thus decreasing the contents of other branched chain amino acids, and as a result, D-alloisoleucine having a high purity, a protected compound thereof and a salt thereof can be obtained. Accordingly, in the method of the present invention, L-isoleucine obtained by fermentation method and the like, which has a low purity, can be used as a starting material.

Step (b): A Step of Conducting Intramolecular Dehydration Condensation of L-(N-carbamoyl)isoleucine to give L-5-sec-butylhydantoin

Intramolecular dehydration condensation of L-(N-carbamoyl)isoleucine can be performed by heating L-(N-carbamoyl)isoleucine, obtained in step (a), in water under acidic conditions, whereby L-5-sec-butylhydantoin can be obtained.

Water to be used as a solvent is generally used in a 1.5- to 20-fold, preferably 2.5- to 10-fold amount, in weight ratio relative to L-(N-carbamoyl)isoleucine.

Intermolecular dehydration condensation is conducted using, for example, an acidic compound such as hydrochloric acid, sulfuric acid, phosphoric acid and the like, under acidic conditions.

For intramolecular dehydration condensation, heating is performed generally at 20° C.—reflux temperature, preferably 80° C.—reflux temperature, preferably for 5 min-5 hrs.

After the completion of step (a), the reaction mixture is adjusted to pH −1 to 5, preferably −0.5 to 0.5, with an acidic aqueous solution (e.g., hydrochloric acid and the like) and the precipitated L-(N-carbamoyl)isoleucine crystal is used for the next step without extraction by filtration and the like, whereby the intramolecular dehydration condensation can be performed only by heating at a temperature in the above-mentioned range.

After the completion of the intramolecular dehydration 10, condensation, the reaction solution is allowed to cool to room temperature for precipitation of L-5-sec-butylhydantoin as crystals. The crystals are collected by filtration, washed with a small amount of water and dried to give crystals of L-5-sec-butylhydantoin.

Step (c): A Step of Conducting an Enzyme Reaction of L-5-sec-butylhydantoin with Hydantoin Racemase (HRase), D-hydantoinase (D-HHase) and N-carbamoyl-D-amino acid hydrolase (D-CHase) to give D-alloisoleucine

The L-5-sec-butylhydantoin obtained in step (b) is subjected to an enzyme reaction with hydantoin racemase (HRase), D-hydantoinase (D-HHase) and N-carbamoyl-D-amino acid hydrolase (D-CHase) to give D-alloisoleucine.

The hydantoin racemase is an enzyme that acts on a 5-substituted hydantoin compound, and catalyzes a reaction to produce a D,L-5-substituted hydantoin compound by racemization of this substance. In the following, it is also indicated as “HRase”.

The D-hydantoinase is an enzyme that acts on a D-5-substituted hydantoin compound, and catalyzes a reaction to produce N-carbamoyl-D-amino acid by hydrolysis of this substance. It is also called D-hydantoin hydrolase. In the following, it is also indicated as “D-HHase”.

The D-carbamoylase is an enzyme that acts on N-carbamoyl-D-amino acid, and catalyzes reaction to produce D-amino acid by hydrolysis of this substance. It is also called N-carbamoyl-D-amino acid hydrolase. In the following, it is also indicated as “D-CHase”.

Preferably, in step (c), transformed cells obtained by introducing a recombinant DNA incorporating HRase, D-HHase and D-CHase are cultured in a medium to give a culture medium where HRase, D-HHase and D-CHase enzymes have been generated, and L-5-sec-butylhydantoin is added to the culture medium to produce D-alloisoleucine. The form of the culture medium is not particularly limited as long as these specific enzymes are contained. In other words, a desired D-alloisoleucine can be produced by directly adding L-5-sec-butylhydantoin to a medium containing the transformant, or mixing L-5-sec-butylhydantoin with a transformant such as microbial cells separated from a medium and the like, a washed transformant, a treated product of transformant obtained by a rupture or melting treatment, a crude enzyme solution that has recovered HHase and the like, an enzyme solution purified further and the like (e.g., WO03/085108). Enzyme reactions using these can be carried out by standing or stirring a reaction mixture containing L-5-sec-butylhydantoin and a culture solution, a separated microbial cell, a washed microbial cell, a microbial cell treated product, a crude enzyme solution or a purified enzyme at a suitable temperature of 25-40° C. (preferably 30-37° C.) while maintaining at pH 5-9 (preferably 7.2-7.7) for 8 hrs-5 days (preferably 24 hrs-72 hrs).

For example, by treating a reaction mixture after the enzyme reaction as in the following, D-alloisoleucine can be extracted.

A reaction mixture after the enzyme reaction is heated, separated by centrifugation, and a 50% aqueous benzalkonium chloride solution is added to a supernatant of an enzyme reaction mixture obtained by centrifugal precipitation. Protein is aggregated with stirring under heating and activated carbon is added for adsorption. Insoluble materials are removed by filtration and the like and the obtained filtrate is concentrated as necessary by, for example, a crystallizing means such as cooling crystallization, neutralization crystallization and the like to allow for crystallization of crude crystals of D-alloisoleucine. The crystals can be further purified by applying crystallization again.

The D-alloisoleucine obtained in step (c) may be subjected to protection of amino group and/or carboxyl group by a conventional method. When either an amino group or a carboxyl group of the obtained protected compound of D-alloisoleucine is not protected, a salt can be formed by a conventional method, whereby a salt of a protected compound of D-alloisoleucine can be also obtained.

As a protecting group for D-alloisoleucine, those generally used for peptide synthesis can be used. As a protecting group for amino group, benzyl group, benzyloxycarbonyl group, tert-butoxycarbonyl group, methoxycarbonyl group, phthaloyl group and the like can be mentioned. As a protecting group for carboxyl group, alkyl groups such as methyl group, ethyl group and the like can be mentioned.

As a salt when forming a salt and when amino group is protected, amines such as dicyclohexylamine, cyclohexylamine and the like; alkali metals such as sodium, potassium and the like; alkaline earth metals such as magnesium, calcium and the like; and the like can be mentioned.

### EXAMPLES

The present invention is explained in detail in the following by referring to examples. The examples are mere exemplifications and do not limit the present invention in any way.

The reaction yield of Examples 1 and 2 was obtained by high performance liquid chromatography (column: Inertsil ODS-3 (4.6φ×150 mm) manufactured by GL Sciences, Inc., temperature 40° C., eluant composition: 50 mM aqueous NaH₂PO₄ solution (pH 2.8): CH₃CN=93:7, flow: 1.0 ml/min, detection: UV 210 nm).

### Example 1

Water (69 ml) was added to L-isoleucine for medicine (13.80 g, 105 mol) and the mixture was heated to 80° C. under stirring. Thereto was added dropwise over 2 hrs an aqueous solution of sodium cyanate (8.19 g, 113 mmol assuming purity to be 90%) dissolved in water (82 g). After the completion of the dropwise addition, the mixture was maintained at said temperature for 30 min, cooled to 50° C. and adjusted to pH 0.18 with 35% hydrochloric acid. Crystals of L-(N-carbamoyl)isoleucine were precipitated while adjusting the pH, thus achieving a slurry state. Subsequently, the slurry was heated to 90° C. and maintained under stirring for 1 hr. Finally, the solid in the slurry was all dissolved to achieve a solution state. This solution was allowed to cool to room temperature to conduct cooling crystallization to give a slurry of L-5-sec-butylhydantoin. This was filtered, washed with a small amount of water and dried at 40° C. to give 15.75 g of crystals. The purity was analyzed to be 97.5% and the yield to the starting material L-isoleucine was 93.6%.

### Example 2

Water (244 ml) was added to crude crystals of L-isoleucine (48.7 g, detailed contents; L-isoleucine content: 92.7%, L-valine content: 0.33%, L-leucine content: 0.29%), and the mixture was heated to 80° C. under stirring. Thereto was added dropwise over 2 hrs an aqueous solution of sodium cyanate (26.81 g, purity 90%) dissolved in water (268 g).

After the completion of the dropwise addition, the mixture was maintained at said temperature for 30 min, cooled to 50° C. and adjusted to pH 0.07 with 35% hydrochloric acid. Crystals of L-(N-carbamoyl)isoleucine were precipitated while adjusting the pH, thus achieving a slurry state. Subsequently, the slurry was heated to 90° C. and maintained under stirring for 1 hr. Finally, the solid in the slurry was dissolved to achieve a solution state by removing the clouding considered to have been derived from impurities in the starting material. This solution was allowed to cool to room temperature to conduct cooling crystallization to give a slurry of L-5-sec-butylhydantoin. This was filtered, washed with water (a little over 80 ml) and air-dried at room temperature to give crude crystals of L-5-sec-butylhydantoin (52.49 g). The purity was analyzed to be 91.3% and the yield to the starting material L-isoleucine was 89.2%. On the other hand, the contents of hydantoin derivatives corresponding to L-valine and L-leucine were 0.14% and 0.13%, respectively, and 57% and 53% were weeded out, respectively.

### Example 3

Culture of Recombinant E. coli

A recombinant E. coli D-9 cell line (described in WO03/085108) containing a hydantoin racemase gene, a D-hydantoinase gene and a D-carbamoylase gene was cultured by the following method.

A D-9 cell line was cultured on an LB agar plate (containing ampicillin 100 mg/L and chloramphenicol 50 mg/L) at 30° C. for 24 hrs.

The obtained microbial cells were inoculated to 50 ml of an LB liquid medium (Sakaguchi flask), and shake cultured (120 rpm) at 30° C. for 16 hrs. The obtained culture solution (1 ml) was transferred to an enzyme producing medium I (300 ml, D-glucose 25 g/L, MgSO₄ 1 g/L, (NH₄)₂SO₄ 5 g/L, KH₂PO₄ 1.39 g/L, FeSO₄.7H₂O 20 mg/L, MnSO₄.5H₂O 20 mg/L, trisodium citrate-2H₂O 2.28 g/L, thiamine.HCl 1 mg/L, pH 7.0) and aerobic culture was performed under stirring at 33° C. for 19 hrs at pH 7.0 (pH control with ammonia gas).

After confirmation of the absence of residual sugar, the culture solution (15 ml) was transferred to an enzyme producing medium II (285 ml, D-glucose 26.3 g/L, MgSO₄ 1.05 g/L, (NH₄)₂SO₄ 5.26 g/L, H₃PO₄ 0.32 g/L, FeSO₄.7H₂O 52.6 mg/L, MnSO₄.5H₂O 52.6 mg/L, trisodium citrate.2H₂O 2.4 g/L, thiamine.HCl 1.05 mg/L, pH 7.0) and aerobic culture was performed under stirring at 33° C. for 9 hrs at pH 7.0 (pH controlled with ammonia gas). After culture for 9 hrs and after confirmation of the absence of residual sugar, a 50% glucose solution was fed to the culture solution at a flow rate of 3.5 ml/h and the aerobic culture was further continued under stirring for 16 hrs at pH 7.0, whereby a recombinant E. coli was obtained (dry cell weight 40 g/L).

### Example 4

The culture solution (60 ml) obtained in Example 3 was added to a substrate solution (540 ml, L-5-sec-butylhydantoin 18 g, KH₂PO₄ 0.41 g, K₂HPO₄ 1.46 g, MnSO₄.5H₂O 145 mg) obtained in Example 2, and the mixture was allowed to react at 37° C. for 48 hrs at pH 7.4 (pH controlled with 1N NaOH, 5N CH₃COOH). After the enzyme reaction, the enzyme reaction mixture was heated to 110° C. for 10 min and subjected to centrifugal separation (8000 g×10 min).

### Example 5

A 50% aqueous benzalkonium chloride solution (1.0 g) was added to a supernatant (593 g, D-alloisoleucine content: 2.27%) of the enzyme reaction mixture obtained by centrifugal precipitation and the mixture was maintained under stirring at 60° C. for 30 min to allow for protein aggregation. Then, activated carbon (5.1 g) was added, and adsorption was conducted at room temperature for 30 min. The slurry was filtered through a filter paper No. 5C and washed with a small amount of water. The thus-obtained filtrate (606 g) was subjected to concentration crystallization under heating (60° C.) under reduced pressure to give a slurry (99.6 g). This was further cooled to 5° C. to allow for crystallization, and the precipitated crystals were filtered and vacuum dried. The weight of the obtained crystals was 11.27 g (D-alloisoleucine content: 97.0%, D-valine content: 0.07%, D-leucine content: 0.08%), and the yield of D-alloisoleucine from the enzyme reaction mixture was 86.7% and the optical purity was not less than 99-0.9% e.e.

The crude crystals of D-alloisoleucine (10.95 g, 80.9 mmol) obtained above was dissolved in 4N aqueous sodium hydroxide solution (23.31 g, 80.9 mmol) and benzyloxycarbonyl chloride (15.19 g, 89.0 mmol) and 4N aqueous sodium hydroxide solution (27.98 g, 97.1 mmol) were simultaneously added dropwise at a constant speed over 30 min while vigorously stirring the mixture in an ice bath. Then the stirring was further continued for 2 hrs. The by-produced small amount of black insoluble material was filtered off, the material was washed with a small amount of water and the filtrate was transferred to a separatory funnel, to which toluene (16 ml) was added for washing. After layer separation, the aqueous layer was removed, 6N hydrochloric acid was added under ice-cooling and pH was lowered to near 2.5, whereby an oily substance was separated. This was extracted with ethyl acetate (100 ml) and washed with 25% brine. Anhydrous sodium sulfate (2 g) was added and the mixture was dried. The anhydrous sodium sulfate was filtered off and dicyclohexylamine (14.68 g, 80.9 mmol) was added dropwise to the ethyl acetate solution. However, the slurry viscosity rose after the middle and stirring became defective. Thus, ethyl acetate (60 ml) was added on the way and dropwise addition was continued. The resulting N-benzyloxycarbonyl(Z)-D-alloisoleucine-dicyclohexylammonium salt was separated, washed with ethyl acetate (20 ml) and vacuum dried at 40° C. to give 31.1 g of crystals. The Z-D-alloisoleucine content was 58.9% relative to the calculated value of 59.4% and the molar yield relative to the starting material D-alloisoleucine was 85.2%. The optical purity was not less than 99.9% e.e.

### INDUSTRIAL APPLICABILITY

According to the present invention, D-alloisoleucine having a high purity, a protected compound thereof and a salt thereof can be produced efficiently, and this method is industrially useful.

This application is based on patent application No. 2004-071577 filed in Japan, the contents of which are hereby incorporated by reference.

1. A production method of D-alloisoleucine, a protected compound thereof or a salt thereof, which comprises steps (a) to (c):
(a) carbamoylating L-isoleucine to give L-(N-carbamoyl)isoleucine,
(b) conducting intramolecular dehydration condensation of L-(N-carbamoyl)isoleucine to give L-5-sec-butylhydantoin, and
(c) conducting an enzyme reaction of L-5-sec-butylhydantoin with hydantoin racemase (HRase), D-hydantoinase (D-HHase) and N-carbamoyl-D-amino acid hydrolase (D-CHase) to give D-alloisoleucine.

2. The method of claim 1, further comprising protecting the D-alloisoleucine obtained in step (c) to give a protected compound of D-alloisoleucine.

3. The method of claim 1, further comprising protecting the D-alloisoleucine obtained in step (c) and forming a salt of the protected compound of D-alloisoleucine to give a salt of the protected compound of D-alloisoleucine.
